(19) United States
(12) Patent Application Publication
(10) Pub. No.: US 2013/0325048 A1
(43) Pub. Date: Dec. 5, 2013

(54) Laparoscopic Manipulation

(71) Applicant: Mark Weiman, Coatesville, PA (US)

(72) Inventor: Mark Weiman, Coatesville, PA (US)

(21) Appl. No.: 13/485,066

(22) Filed: May 31, 2012

(51) Int. Cl. A61B 17/00; A61B 17/32

(52) U.S. Cl. 606/170; 606/1

(57) ABSTRACT

A surgical instrument for use in a therapeutic treatment of a patient has a stem connected to a handle, and a slide connected to a trigger. An end effector forms a distal portion for manipulating body tissue, for example a curette, and a proximal portion that is pivotally connected to either the stem or slide. A lever extends from near the pivot and is engaged by the other of the stem or slide. When the slide and stem are slid relative to each other, the lever is engaged to pivot the end effector within the body, for example to cut tissue. A post on the lever engages a channel in the stem or slide, to guide movement of the end effector. A link may be pivotally connected between the lever and a stem or slide, to increase a range of motion of the end effector.

### FIELD OF THE INVENTION

The invention relates to manipulating an end effector within the body, and more particularly to a laparoscopic instrument having an end effector such as a curette having a wide angle of movement.

### BACKGROUND OF THE INVENTION

Bones and bony structures are susceptible to a variety of weaknesses that can affect their ability to provide support and structure. Weaknesses in bony structures have numerous potential causes, including degenerative diseases, tumors, fractures, and dislocations. Advances in medicine and engineering have provided doctors with a plurality of devices and techniques for alleviating or curing these weaknesses.

In some cases, the spinal column requires additional support in order to address such weaknesses. One technique for providing support is to extend a structure between adjacent bones, the structure connected at each end to a polyaxial screw “tulip”, or yoke, connected to a bone screw inserted within the bone.

Preparation of the intervertebral disc space includes maneuvering a cutting tool within a small space. Access to the disc space may be limited, as well, for example when conducting a minimally invasive procedure. The foregoing and other diseases often require the removal or shaping of body tissue through a minimal incision, for example in a laparoscopic procedure, where the tool must be sufficiently small to pass through a small opening in the body.

### SUMMARY OF THE INVENTION

In accordance with an embodiment of the disclosure, a surgical instrument for use in a therapeutic treatment of a patient, comprises a stem; a slide slideably connected to the stem; an end effector having a proximal portion and a distal portion, the distal portion configured to provide a useful function inside the patient, the proximal portion pivotally connected to one of the stem or the slide at a first pivot; a lever extending from about the proximal end of the end effector at a proximal end of the lever, the lever having a distal end engageable by the other of the stem or the slide at a first pivot; a lever extending from about the proximal end of the end effector at a proximal end of the lever, the lever having a distal end engageable by the other of the stem or the slide relative to the first pivot, the end effector pivoted about the first pivot when the lever is engaged by the other of the stem or the slide.

In various embodiments thereof, the slide and the stem are less than 15 mm wide when connected; the instrument is sized and dimensioned for laparoscopy; the connected stem and slide are sized and dimensioned to be insertable into the patient through a minimally invasive puncture in the skin of the patient, and the end effector is not substantially wider than the connected stem and slide; the instrument further includes a handle connected to the stem, and a trigger connected to the slide; and the instrument further includes a biasing element operative to move the trigger from a triggered position to a resting position.

In further embodiments thereof, the distal end of the lever includes an extending post, and the other of the engageable stem or slide is provided with a channel through which the extending post moves as the slide and the stem are slid relative to each other, movement of the extending post within the guide operative to pivot the end effector about the first pivot.

In an additional embodiment thereof, a distal end of the lever is extendable through an opening in the other of the engageable stem or slide; the end effector pivots through an angle of at least 145 degrees; the end effector pivots between an angle substantially parallel to a longitudinal axis of the instrument to an angle of 145 degrees with respect to the longitudinal axis; the instrument further includes a link pivotally connected to the distal end of the lever at one end of the link, and pivotally connected to the other of the stem or the slide at an opposite end of the link; the other of the stem or the slide is the stem.

In yet further embodiments, the instrument further includes a shaped portion formed upon an exterior surface of the stem or the slide to which the end effector is pivotally connected, the shaped portion sized and dimensioned to receive a portion of the link; the end effector is a curette; the end effector is selected from the group consisting of cutter, curette, pincher, grasper, light source, dilator, clamp, hose, retractor, sensor, other articulating tool, and optical device; and the end effector is moved through non-circular path as the stem is slid relative to the slide.

In another embodiment of the disclosure, a laparoscopic surgical instrument for use in a therapeutic treatment of a patient, comprises a first elongate extension; a second elongate extension slideably connected to the first elongate extension and having a distal end including a channel; an end effector having a proximal portion and a distal portion, the distal portion configured to provide a useful function inside the patient, the proximal portion pivotally connected to the first elongate extension at a first pivot; and a lever extending from about the proximal end of the end effector at a proximal end of the lever, the lever having a distal end including a post, the post engageable in the channel whereby the end effector is pivoted about the first pivot when the first and second elongate extensions are slid relative to each other to cause the post to move within the channel.

In embodiments thereof, the surgical instrument further includes a handle connected to one of the first and second extensions, and a trigger connected to the other of the first and second extensions; and the end effector is selected from the group consisting of cutter, curette, pincher, grasper, light source, dilator, clamp, hose, retractor, sensor, other articulating tool, and optical device.

Further in accordance with the disclosure, a method of manipulating tissue inside a body of a patient, comprises inserting slidably connected first and second elongate extensions into a minimally invasive opening in the body of the patient, the second elongate extension having a distal end including a channel; and sliding the first elongated extension relative to the second elongated extension to move an end effector through a path of travel, the end effector having a proximal portion and a distal portion, the distal portion configured to provide a useful function inside the patient, the proximal portion pivotally connected to the first elongate extension at a first pivot, the end effector having a lever extending from about the proximal end of the end effector at a proximal end of the lever, the lever having a distal end including a post, the post engageable in the channel whereby sliding the first elongated extension relative to the second elongated extension causes the post to move within the channel and guide a movement of the lever and end effector.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an illustration of a tool in accordance with the disclosure, with an end effector aligned with a longitudinal axis of the tool;

FIG. 2 depicts the tool of FIG. 1, with the end effector positioned at a small angle with respect to the longitudinal axis of the tool;

FIG. 3 illustrates an enlarged view of the distal, or end effector end of the tool of FIG. 1;

FIG. 4 depicts the distal end of FIG. 3, with the end effector forming an angle with respect to a longitudinal axis of the tool;

FIG. 5 depicts the distal end of the tool of FIG. 2;

FIGS. 6-8 are cross-sections through a longitudinal midline of the distal tool ends of FIGS. 2-4;

FIGS. 9 and 11 are perspective views of the distal tool end of the FIG. 3;

FIG. 10 is a perspective view of the distal tool end of FIG. 4;

FIG. 12 is a side view of a distal tool end of an alternative tool in accordance with the disclosure;

FIG. 13 depicts the distal tool end of FIG. 12, with an end effector at a small angle relative to a longitudinal axis of the tool;

FIGS. 14-15 are perspective views of the distal tool ends of FIGS. 12 and 13, respectively;

FIG. 16 is an illustration of the tool corresponding to the distal tool end of FIG. 12;

FIG. 17 is an alternative tool in accordance with the disclosure;

FIG. 18 is an exploded view of the tool of FIG. 17;

FIG. 19 is a cross-section of the tool of FIG. 17, taken along the line “A-A” of FIG. 22;

FIG. 20 depicts the tool of FIG. 17, with an end effector retracted within the tool;

FIG. 21 depicts the tool of FIG. 17, with an end effector extended to an operational position; and

FIG. 22 is a side view of the tool of FIG. 20.

### DETAILED DESCRIPTION OF THE INVENTION

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality, as used herein, is defined as two or more than two. The term another, as used herein, is defined as at least a second or more. The terms “including” and “having,” as used herein, are defined as comprising (i.e., open language).

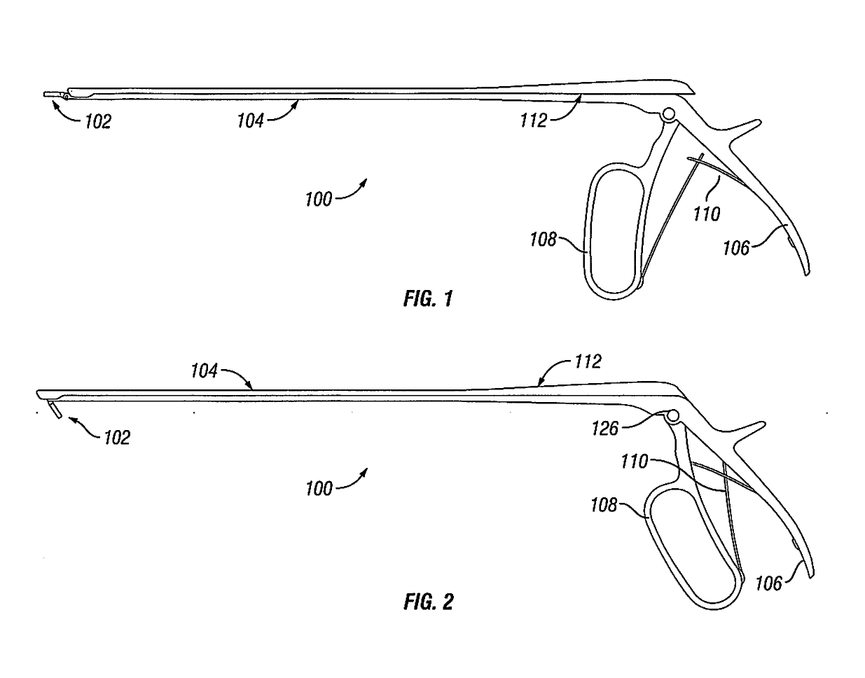

With reference to FIG. 1, a surgical instrument, or tool 100 includes an articulating end effector 102, in this embodiment a ring curette for cutting tissue. For example, tool 100 may be used to prepare vertebral endplates, or an intervertebral space, for insertion of a stabilizing implant. Tool 100 enables a large angle of articulation of end effector 102, which is advantageously a curette, but may have any shape known or hereinafter developed. Moreover, tool 100 may be used to articulate one or more of any type of device, including but not limited to the examples of cutter, curette, pincher, grasper, light source, dilator, clamp, hose, retractor, sensor, other articulating tool, or optical device. Tool 100 further advantageously includes an extension 104, a handle 106 connected to extension 104, a moveable trigger 108, and a trigger bias 110 operative to return trigger 108 to a starting position after actuation.

While a surgical instrument particularly benefits from the disclosure, it should be understood that any tool requiring a wide range of motion, insertable through a small opening, may be fabricated in accordance with the disclosure. This may include, for example, mechanical repair or assembly tools.

Extension 104 includes a stem 112 and a slide 114 which are mutually connected whereby stem 112 is affixed to handle 106, and slide 114 is caused to slide relative to stem 112 as trigger 108 is actuated or operated. Stem 112 and slide 114 may be connected by any known means, including a dovetail or other mutually interlocking configuration, or stem 112 may form an enclosure or sleeve containing slide 114.

In accordance with the disclosure, stem 114 and slide 112 are elongated and narrow, so that they may form a laparoscopic instrument passable into the body of a patient through a minimally invasive incision, for example though a stab type incision which may be less than 30 mm in length, for example about 15 mm in length, but may be larger as deemed therapeutically best by the medical practitioner. Typically, such instruments form a total width of less than 15 mm, and in most cases substantially less than 15 mm, for example about 6 mm or less. To pass tool 100 into the body, it is accordingly advantageous that end effector 102 is not wider than the combined stem 114 and slide 112.

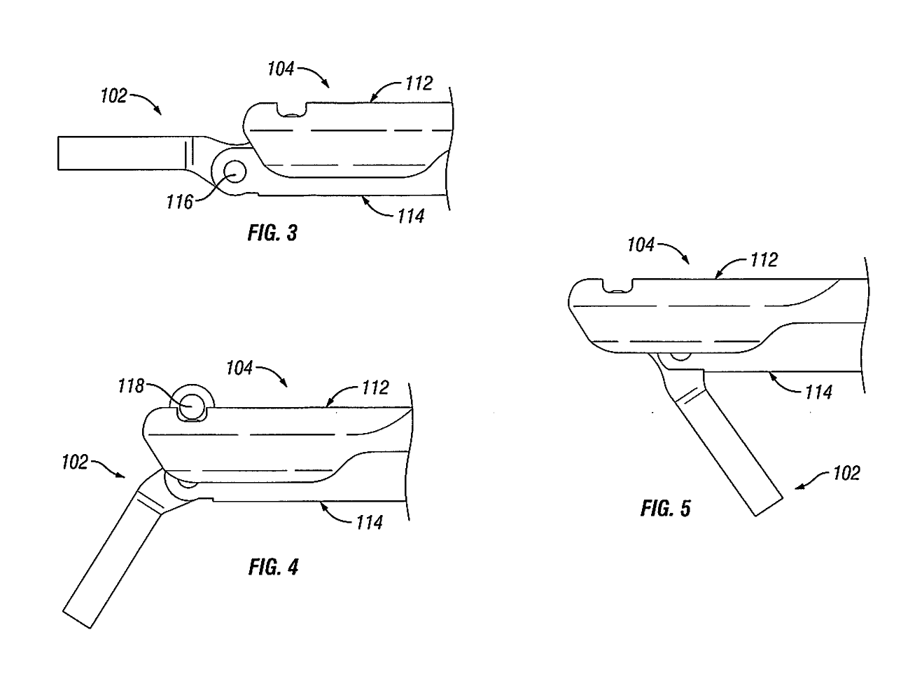

With reference to FIGS. 3-5, end effector 102 pivots about an effector pivot pin 116 mounted at a distal end of slide 114. End effector 102 is positionable to extend along a longitudinal axis of extension 104, forming a 180 degree angle with extension 104, as shown in FIG. 3, to be insertable through an opening substantially as small as an opening required for extension 104. In FIG. 4, illustrates an angle of about 120 degrees, and FIG. 5 illustrates an angle of about 55 degrees, although a more acute angle may be formed. To extend end effector 102 through an area defined as 360 degrees minus two times the smallest angle formable, the entirety of tool 100 may rotated 180 degrees when end effector is extended to 180 degrees, and end effector may be thereafter once again pivoted through its full range of motion. Tool 100 may of course be angled or translated in order for end effector 102 to reach areas that do not lie within this complete range of motion. A wider angle is achievable in accordance with the disclosure, including for example a range of motion of end effector 102 of about 145 degrees or more with respect to a longitudinal axis through the longest dimension of tool 100.

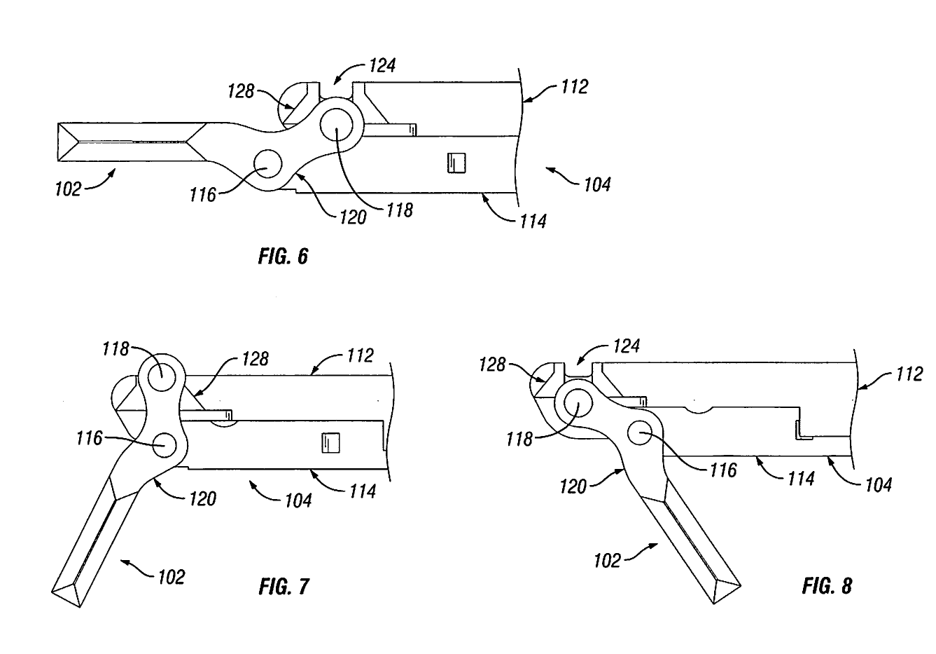

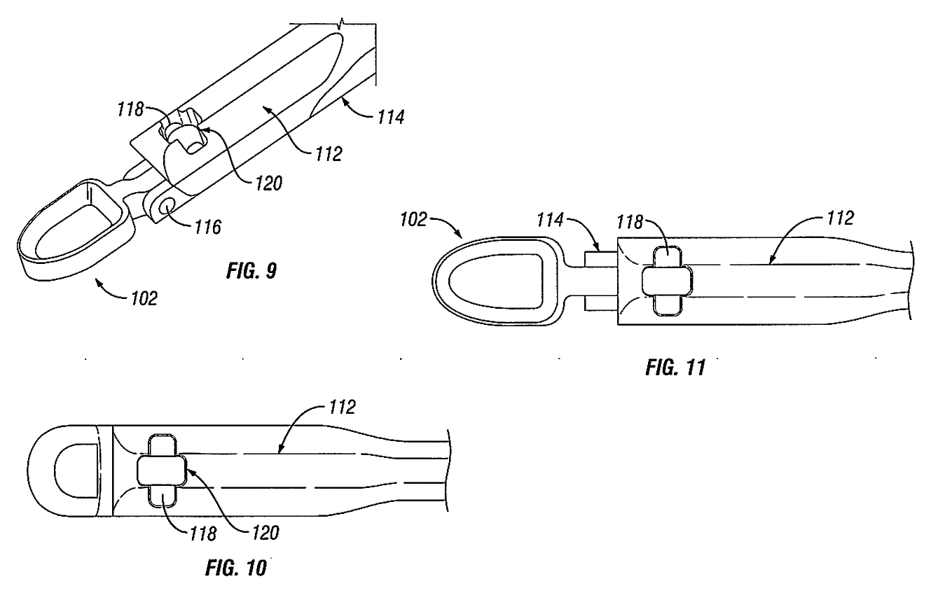

Reference may be had to FIGS. 6-8, where an interior of extension 104 and end effector 102 are illustrated in cross section, and an operation of slide 114 and associated elements may be understood. Following pin or post 118 is inserted within an end effector lever 120 to extend laterally therefrom on at least one side, to thereby engage guide channel 124, which may be formed within either or both sides of stem 112. In FIG. 6, post 118 is located within a lower or inner extent of guide channel 124, thereby disposing end effector 102 at 180 degrees with respect to extension 104.

In FIG. 7, slide 114 is moved in the direction of the proximal, or handle end of tool 100. Such movement may be carried out by moving trigger 108 towards a distal end of tool 100, pivoting trigger 108 about Trigger pivot pin 126. It should be understood that a gear, cam, or other intermediate force transmitting structure may be used to cause an opposite movement of slide 114, as desired. As slide 114 moves proximally, post 118 is urged along guide channel 124, levering end effector to a smaller angle with respect to extension 104. In the embodiment shown, post 118 emerges from an interior of stem 112, to enable sufficient travel of post 118 to maximize an extent of leverage and movement of end effector 102, although such extent is not a requirement in accordance with the disclosure. One or more channels 128 may be formed to permit movement of lever 120 within an interior of stem 112.

In FIG. 8, it may be seen that slide 114 has been drawn further proximally and a yet smaller angle is formed between end effector 102 and extension 104. To enable a greater range of motion in this direction, post 118 descends within guide channel. Angles smaller than that shown in the figures are possible, dependent for example upon factors including but not limited to a thickness of end effector 102, slide 114, effector pivot pin 116, and cam follower post 118, or a length of lever 120.

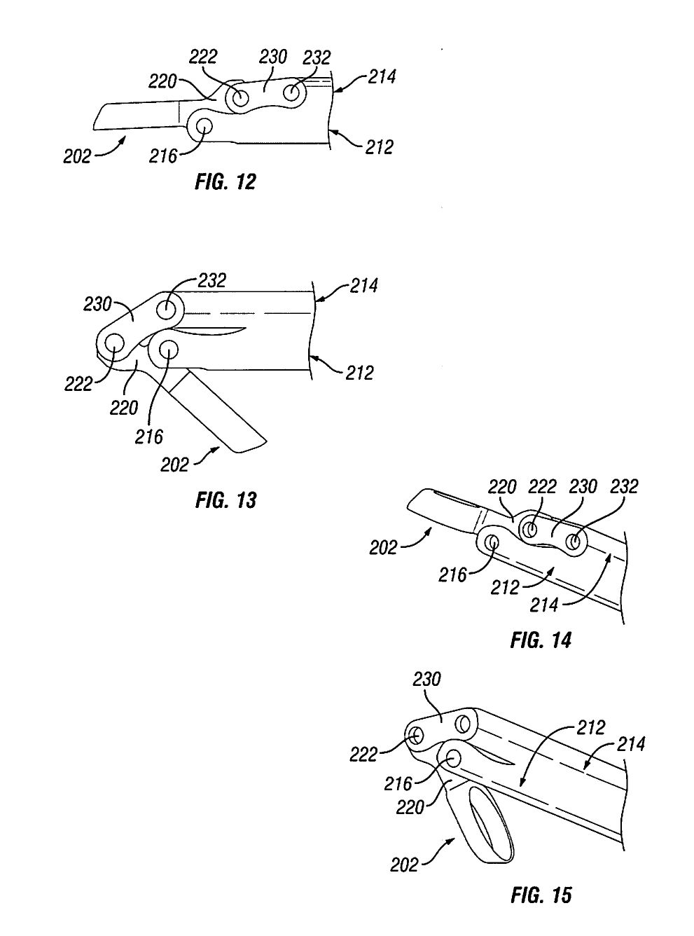

In an alternative embodiment of the disclosure, tool 100A, shown in FIGS. 12-16, includes end effector 202 which pivots upon effector pivot pin 216 affixed to stem 212. An end effector lever 220 extends from pin 216 and end effector 202, at a proximal end, to an intermediate pivot pin 222 at a distal end. A linkage 230 joins intermediate pivot pin 222 to slide 114 at a slide pivot pin 232. Tool 100A is configured to be passable through a minimally invasive incision in the manner as described for tool 100, and to provide a like range of motion for end effector 102.

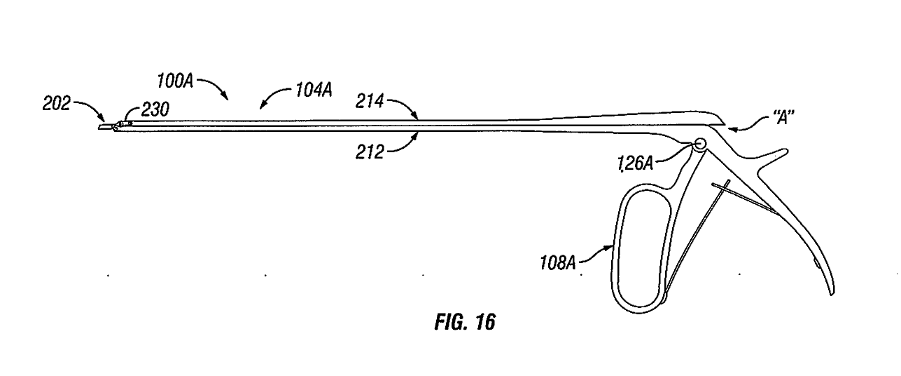

With reference to FIG. 16, slide 214 is positioned, in this embodiment, above stem 212, with respect to trigger 108A. Trigger pivot pin 126A is affixed to stem 212, and trigger 108A pivots thereupon in engagement with slide 214. As with trigger 108, trigger 108A contacts slide 214 in a pivoting or socketed connection, not shown, whereby an end of trigger 108A may pivot while driving slide 214, in a manner that would be understood within the art. It should be understood, however, in accordance with the disclosure, slide 114 or 214 may be positioned above or below stem 112 or 212, respectively. In FIG. 16, it may be seen at reference “A” that slide 214 has been moved proximally, and overhangs stem 212.

In use, as slide 214 is moved distally with respect to trigger 108A, linkage 230 is pushed distally, pivoting lever 220 at intermediate pivot pin 222 and end effector pivot pin 216, causing end effector 202 to rotate about end effector pivot pin 216. As slide 214 continues distally, intermediate pivot pin 222 passes beyond an extent of stem 212, enabling linkage 230 to pivot about slide pivot pin 232, enabling additional excursion of end effector 202, thereby enabling a smaller angle to be formed between end effector 202 and stem 214. Differing angular dispositions of end effector 202 are shown in perspective, in FIGS. 14-16.

When slide 214 is moved proximally to a starting position, the portion of link 230 that is widened to accommodate intermediate pivot pin 222 is positioned to rest within a shaped portion of stem 212, thereby enabling a smaller profile of tool 100A. It should also be realized that a mating profile formed between stem 212 and linkage 230 may be used to govern a path of travel of end effector 202 that is non-regular.

More particularly, a path of end effector 202 may be formed that is non-circular, and instead, is elliptical or widened over a portion of an arc. This is advantageous, for example, when end effector is a curette, and a space is being formed within an intervertebral disc space for insertion of a spacer. The vertebral body may be wider laterally than it is in an anterior/posterior dimension, and therefore a widened cutting path may be advantageous.

It should be understood that while connections are described with respect to a slide or a stem, it is the relative movement of the stem and slide which causes engagement of the lever and movement of the end effector through a path. Accordingly, references to slide 114/214 or stem 112/212 may be interchanged throughout the disclosure.

While tool 100/100A is illustrated with a manually operable handle and trigger 108/108A, it should be understood that extension 104/104A may be connected to other manual or automated actuators, including robotic manipulators, or handheld devices, for example a grip with a thumb actuator (not shown), which may be manually, pneumatically, hydraulically, or electrically actuated to move slide 114/214 relative to stem 112/212.

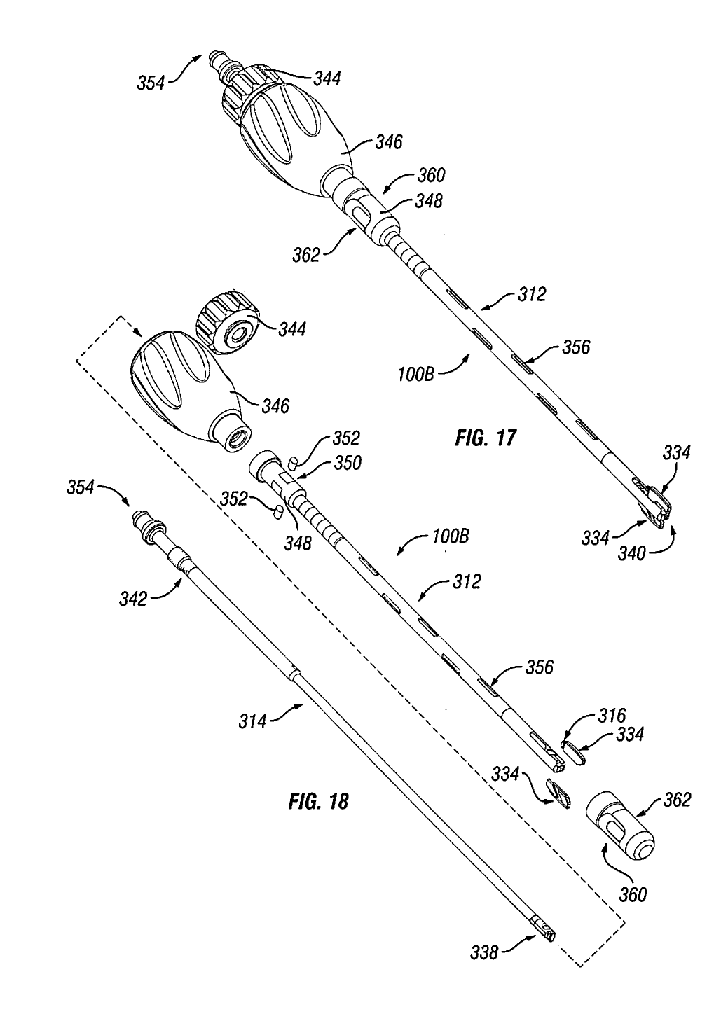

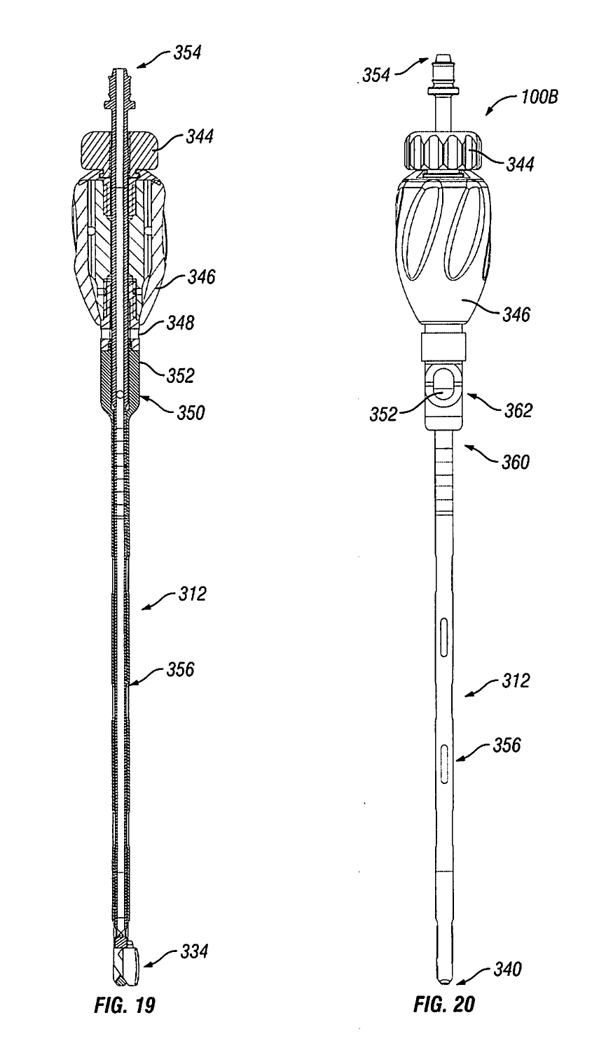

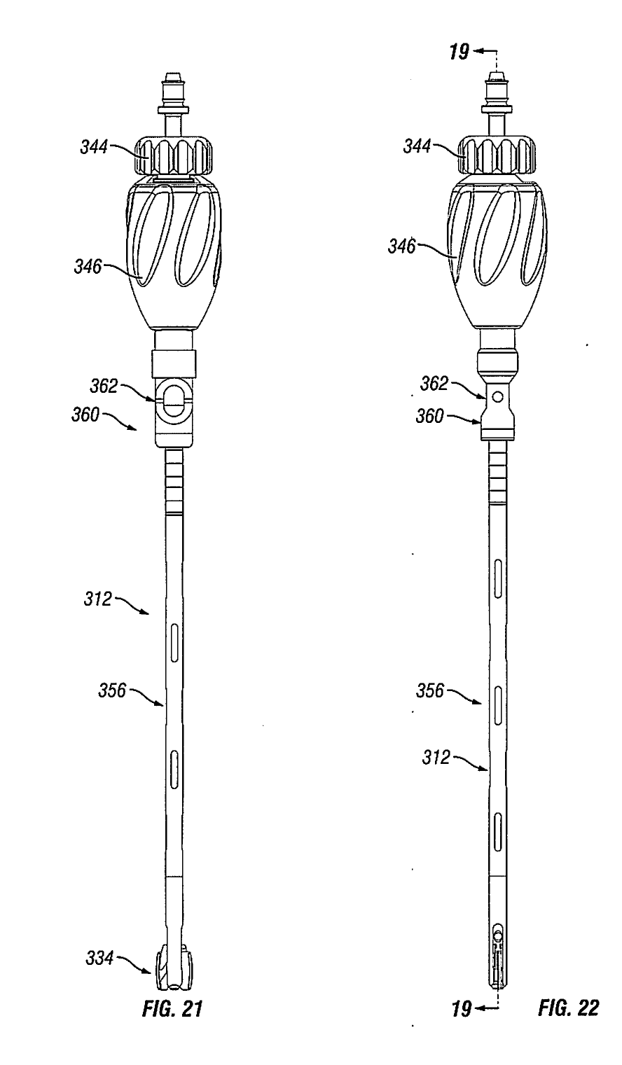

With reference to FIGS. 17-18, an expanding tool 100B includes a stem 312 moveably supporting an end effector 302 having a plurality of moveable elements 334. In one embodiment, moveable elements 334 pivot about element pin 316 at a proximal end in connection with stem 312. Moveable elements 334 are advantageously biased by a biasing element, such as a spring (not shown), to pivot towards each other. In this manner, in a first position, moveable elements 334 are contained within stem 312, as shown in FIG. 20. A second position, in which moveable elements are separated relative to each other, is shown in FIG. 17. To move moveable elements 334 from the first position to the second position, a slide 314 is advanced along stem 312 until a distal end 336 of slide 314 passes between, and separates, moveable elements 334.

Slots 336 in stem 312 admit passage of moveable elements 334 from an interior of stem 312 in the first position, to a location at least partially exterior to stem 312 in the second position. In one embodiment, slide 314 includes grooves 338 which engage and support moveable elements 334 throughout all or a portion of a range of motion of slide 314. In another embodiment, grooves 338 are ramped, facilitating separation of moveable elements 334 as slide 314 is advanced towards a distal end 340 of tool 100B. In one embodiment, moveable elements remain parallel as they are separated, due to a cooperating mating engagement surface of moveable elements 334 and slide 314.

To effectuate movement of slide 314, in the embodiment shown, threads 342 disposed upon a surface of slide 314, cooperate with threads internal to knob 344, the latter rotatably retained within a handle, or grip 346. Slide 314 is passed through knob 344, grip 346, and into stem 312, the latter secured to grip 346. A guide tube 348 is affixed to stem 312, and has one or more elongated channels 350 extending longitudinally with respect to a longitudinal axis of tool 100B.

A slide guide 348 is affixed to grip 346 and or stem 312, and is provided with one or more alignment slots 350. Alignment pins 352 are passed through alignment slots 350 and are affixed to slide 314. In this manner, slide 314 is prevented from rotating within stem 312. Knob 344 is threaded onto slide 314, and connected to grip 346 so that knob 344 is free to rotate, but not separate from grip 346. In this manner, as knob 344 is rotated, slide 314 is advanced distally or proximally with respect to stem 312, and may thereby advance or withdraw with respect to moveable elements 334. As slide 314 is advanced, a position of alignment pins 352 may be viewed outside the patient's body, to determine an expansion state of moveable elements 334.

In one embodiment, an indicating sleeve 360 is advantageously, but not necessarily, positioned over slide guide 348 and affixed to stem 312 and or grip 346. Alignment pins 352 are passed through one or more slots 362 in indicating sleeve 360 and alignment slots 350 before being affixed to slide 314. Indicia upon indicator indicating sleeve 360 may be compared with a position of alignment pins 352 within slots 362, to indicate a position of slide 314 within stem 312, and accordingly, a position of moveable elements 334. A visible end of alignment pins 352 may be painted a contrasting color to aid in viewing an indicated position.

In one embodiment, slide 314 is hollow, and forms an aspirating channel within, accessible by an aspiration port 354 at a proximal end of slide 314. Port 354 may be connected to suction or pressure generating apparatus as is known in the art. One or more aspirating apertures 356 may be provided upon stem 312 to enable passage of low or high pressure gas or liquids from port 354 to the patient, to introduce gas or liquids to the patient, or to remove gas, liquids, and or solids from the patient. In one embodiment, moveable elements 334 are cutting blades or rasps, in which case cut or abraded tissue is collected through aspirating apertures 356.

In an embodiment, moveable elements 334 are scraper blades, operative to remove tissue as tool 100B is moved within the body, for example as tool 100B is rotated within a space, such as an intervertebral space. In other embodiments, moveable elements 334 may be a cutter, curette, pincher, grasper, light source, dilator, clamp, hose, retractor, sensor, other articulating tool, or optical device.

Tool 100/100A/100B may be made from any known material, and is advantageously fabricated with materials which are biocompatible, and which may be sterilized using heat or gas, for example. Example materials include metal, such as austenitic or martensitic stainless steel, advantageously heat treated and passivated, or titanium; or plastic, such as high molecular weight polyethylene, polyphenyl-sulfone, acrylic, or polypropylene. It should be understood that these are examples, and any material of suitable biocompatibility, mechanical strength, and durability may be selected. Tool 100/100A/100B of the disclosure may be fabricated using any known or hereinafter developed method, including forging, molding, etching, stamping, or casting.

All references cited herein are expressly incorporated by reference in their entirety. There are many different features to the present invention and it is contemplated that these features may be used together or separately. Unless mention was made above to the contrary, it should be noted that all of the accompanying drawings are not to scale. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention.

### REFERENCE NUMERAL LIST

100 tool
100A tool
110B tool
102 end effector
104 extension
104A extension
106 handle
108 moveable trigger
110 trigger bias
112 stem
114 slide
116 effector pivot pin
118 pin
120 end effector lever
124 guide channel
126 trigger pivot pen
128 channel
202 end effector
212 stem
214 slide
216 pivot pin
220 end effector lever
222 intermediate pivot pin
230 linkage
232 slide pivot pin
302 end effector
312 stem
314 slide
316 element pin
334 moveable element
336 slot
338 groove
340 distal end
342 thread
344 knob
346 grip
348 guide tube
350 elongated channel
352 alignment pin
354 port
356 aspirating aperture
360 indicating sleeve
362 slot

What is claimed is:

1. A surgical instrument for use in a therapeutic treatment of a patient, comprising:
a stem;
a slide slideably connected to said stem;
an end effector having a proximal portion and a distal portion, said distal portion configured to provide a useful function inside the patient, said proximal portion pivotally connected to one of said stem or said slide at a first pivot; and
a lever extending from about said proximal end of said end effector at a proximal end of said lever, said lever having a distal end engageable by the other of said stem or said slide relative to said first pivot, said end effector pivoted about said first pivot when said lever is engaged by the other of said stem or said slide.

2. The surgical instrument of claim 1, wherein said slide and said stem are less than 15 mm wide when connected.

3. The surgical instrument of claim 1, wherein the instrument is sized and dimensioned for laparoscopy.

4. The surgical instrument of claim 1, wherein said connected stem and slide are sized and dimensioned to be insertable into the patient through a minimally invasive puncture in the skin of the patient, and said end effector is not substantially wider than said connected stem and slide.

5. The surgical instrument of claim 1, further including a handle connected to said stem, and a trigger connected to said slide.

6. The surgical instrument of claim 5, further including a biasing element operative to move said trigger from a triggered position to a resting position.

7. The surgical instrument of claim 1, wherein said distal end of said lever includes an extending post, and said other of said engageable stem or slide is provided with a channel through which said extending post moves as said slide and said stem are slid relative to each other, movement of said extending post within said guide operative to pivot said end effector about said first pivot.

8. The surgical instrument of claim 7, wherein a distal end of said lever is extendable through an opening in said other of said engageable stem or slide.

9. The surgical instrument of claim 1, wherein said end effector pivots through an angle of at least 145 degrees.

10. The surgical instrument of claim 1, wherein said end effector pivots between an angle substantially parallel to a longitudinal axis of the instrument to an angle of 145 degrees with respect to the longitudinal axis.

11. The surgical instrument of claim 1, further including a link pivotally connected to said distal end of said lever at one end of said link, and pivotally connected to the other of said stem or said slide at an opposite end of said link.

12. The surgical instrument of claim 11, wherein the other of said stem or said slide is said stem.

13. The surgical instrument of claim 11, further including a shaped portion formed upon an exterior surface of said stem or said slide to which said end effector is pivotally connected, said shaped portion sized and dimensioned to receive a portion of said link.

14. The surgical instrument of claim 1, wherein said end effector is a curette.

15. The surgical instrument of claim 1, wherein said end effector is selected from the group consisting of cutter, curette, pincher, grasper, light source, dilator, clamp, hose, retractor, sensor, other articulating tool, and optical device.

16. The surgical instrument of claim I, wherein said end effector is moved through non-circular path as said stem is slid relative to said slide.

17. A laparoscopic surgical instrument for use in a therapeutic treatment of a patient, comprising:
a first elongate extension;
a second elongate extension slideably connected to said first elongate extension and having a distal end including a channel;
an end effector having a proximal portion and a distal portion, said distal portion configured to provide a useful function inside the patient, said proximal portion pivotally connected to said first elongate extension at a first pivot; and
a lever extending from about said proximal end of said end effector at a proximal end of said lever, said lever having a distal end including a post, said post engageable in said channel whereby said end effector is pivoted about said first pivot when said first and second elongate extensions are slid relative to each other to cause said post to move within said channel.

18. The surgical instrument of claim 17, further including a handle connected to one of said first and second extensions, and a trigger connected to the other of said first and second extensions.

19. The surgical instrument of claim 17, wherein said end effector is selected from the group consisting of cutter, curette, pincher, grasper, light source, dilator, clamp, hose, retractor, sensor, other articulating tool, and optical device.

20. A method of manipulating tissue inside a body of a patient, comprising:
inserting slidably connected first and second elongate extensions into a minimally invasive opening in the body of the patient, the second elongate extension having a distal end including a channel; and
sliding the first elongated extension relative to the second elongated extension to move an end effector through a path of travel, the end effector having a proximal portion and a distal portion, the distal portion configured to provide a useful function inside the patient, the proximal portion pivotally connected to the first elongate extension at a first pivot, the end effector having a lever extending from about the proximal end of the end effector at a proximal end of the lever, the lever having a distal end including a post, the post engageable in the channel whereby sliding the first elongated extension relative to the second elongated extension causes the post to move within the channel and guide a movement of the lever and end effector.
